(19) United States
(12) Patent Application Publication
(10) Pub. No.: US 2023/0232761 A1
(43) Pub. Date: Jul. 27, 2023

(54) CULTIVATION SYSTEMS

(71) Applicant: W. L. Gore & Associates, Inc., Newark, DE (US)

(72) Inventor: Norman E. Clough, Landenberg, PA (US)

(21) Appl. No.: 18/012,282

(22) PCT Filed: Jun. 25, 2021
(86) PCT No.: PCT/US2021/039150

§ 371 Date: Dec. 22, 2022

Related U.S. Application Data
(60) Provisional application No. 63/044,285, filed on Jun. 25, 2020.

(51) Int. Cl. A01G 33/00; C12M 1/12

(52) U.S. Cl. CPC A01G 33/00; C12M 25/02

(57) ABSTRACT

Cultivation systems including a cultivation substrate configured to promote seaweed holdfast formation and seaweed attachment are disclosed. The cultivation systems may include one or more of a nutrient phase, an adhesive, a bioactive agent, a liquid containing phase. The cultivation substrates may be patterned. The cultivation systems may specifically retain and viably maintain specific seaweed species such as dulse, kelp and nori.

### CROSS-REFERENCE TO RELATED APPLICATION

This application is a national phase application of PCT Application No. PCT/US2021/039150, internationally filed on Jun. 25, 2021, which claims the benefit of Provisional Application No. 63/044,285, filed Jun. 25, 2020, which are incorporated herein by reference in their entireties for all purposes.

### FIELD

The present disclosure relates generally to cultivation systems, and more specifically to seaweed cultivation systems configured to support holdfast formation.

### BACKGROUND

The current process to cultivate seaweed from spores involves using textured nylon “culture strings” or “seed strings” to which the spores weakly attach during a lab-based seeding. The culture string containing weakly attached juvenile seaweed (gametophytes and sporophytes) is then wound onto ropes at a seaweed farm, where the ropes are subsequently placed under water. The process is inherently variable in terms of yield and throughput due in large part to biofouling (i.e., the contamination of the seed string with unwanted species of seaweed and other organisms). Biofouling can severely reduce seaweed growth and yields. Traditionally, effective biofouling-resistant materials (smooth, low coefficient of friction films) also reduce seaweed growth and yield due to poor attachment to these substrates. Other factors affecting yield and throughput include the ease by which the seaweed can be damaged from, for example, currents, changes in temperature, and nutrient availability. Further, poor packaging and handling can result in damage and loss of juvenile seaweed. Current approaches to improving stability of juvenile seaweed on culture strings is focused on the surface texture of existing fibers. Indeed, fiber texture of culture strings is very important to the success of seaweed cultivation. There is a need for a substrate that can provide for effective attachment and growth of seaweed while also providing effective anti-biofouling properties.

### SUMMARY

Various embodiments are directed toward cultivation systems configured to retain and viably maintain spores.

According to one example (“Example 1”), a cultivation system includes a cultivation substrate including a low porosity substrate having a porosity of about 10% or less, and a fibrillated submicron surface structure configured to retain seaweed by a holdfast.

According to another example (“Example 2”), further to Example 1, the fibrillated submicron surface structure is characterized by an average inter-fibril distance up to and including 1000 nm.

According to another example (“Example 3”) further to Example 1 or Example 2, the fibrillated submicron surface structure has an average depth of about 1000 nm or less.

According to another example (“Example 4”) further to any one of Examples 1 to 3, the fibrillated submicron surface structure has an average depth of about 5 nm or less.

According to another example (“Example 5”) further to any one of Examples 1 to 4, the low porosity substrate is about 25.4 µm (1 mil) to about 762 µm (30 mil) thick.

According to another example (“Example 6”) further to any one of Examples 1 to 5, the low porosity substrate is about 25.4 µm (1 mil) to about 127 µm (5 mil) thick.

According to another example (“Example 7”) further to any one of Examples 1 to 6, the cultivation substrate is configured as a tape, a substrate, a woven article, a non-woven article, a braided article, a knit article, a fabric, a particulate dispersion, or combinations of two or more of the foregoing.

According to another example (“Example 8”) further to any one of Examples 1 to 7, the cultivation substrate includes at least one of a backer layer, a carrier layer, a laminate of a plurality of layers, a composite material, or combinations thereof.

According to another example (“Example 9”) further to any one of Examples 1 to 8, the low porosity substrate comprises an expanded fluoropolymer.

According to another example (“Example 10”) further to Example 9, the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDFco-(TFE or TrFE)), and expanded polytetrafluoroethylene (ePTFE).

According to another example (“Example 11”) further to any one of Examples 1 to 10, the low porosity substrate is an expanded polytetrafluoroethylene (ePTFE) substrate.

According to another example (“Example 12”) further to any one of Examples 1 to 8, the low porosity substrate comprises an expanded thermoplastic polymer.

According to another example (“Example 13”) further to Example 12, the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

According to another example (“Example 14”) further to any one of Examples 1 to 8, the low porosity substrate comprises an expanded polymer.

According to another example (“Example 15”) further to Example 14, wherein the expanded polymer is expanded polyurethane (ePU).

According to another example (“Example 16”) further to any one of Examples 1 to 8, the low porosity substrate is expanded polyparaxylylene (ePPX).

According to another example (“Example 17”) further to Example 11, the PTFE substrate has a water vapor permeability coefficient of about 0.015 g-mm/m²/day or less, and is formed by a method comprising: (a) preparing a biaxially expanded PTFE film; (b) densifying the expanded PTFE film; and (c) stretching the densified expanded PTFE film.

According to another example (“Example 18”) further to Example 18, the densified expanded PTFE film is stretched at a temperature exceeding the crystalline melt temperature of PTFE in step (c).

According to another example (“Example 19”) further to Example 17 or Example 18, the expanded PTFE film is sintered prior to step (b).

According to another example (“Example 20”) further to any one of claims 17 to 19, the biaxially expanded PTFE film includes two or more plies of expanded PTFE.

According to another example (“Example 21”) further to any one of Examples 17 to 20, steps (a)-(c) are carried out in a continuous manner.

According to another example (“Example 22”) further to any one of Examples 1 to 21, the cultivation substrate further includes a high porosity substrate having a porosity of at least 30%, and a node and fibril microstructure characterized by an average inter-fibril distance of about 1 µm to 500 µm, or an average pore size of about 1 µm to about 500 µm.

According to another example (“Example 23”) further to Example 22, the high porosity substrate comprises an expanded fluoropolymer.

According to another example (“Example 24”) further to Example 23, the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDFco-(TFE or TrFE)), and expanded polytetrafluoroethylene (ePTFE).

According to another example (“Example 25”) further to any one of Examples 22 to 24, the high porosity substrate is an expanded polytetrafluoroethylene (ePTFE) substrate.

According to another example (“Example 26”) further to Example 22, the high porosity substrate comprises an expanded thermoplastic polymer.

According to another example (“Example 27”) further to Example 26, the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

According to another example (“Example 28”) further to Example 22, the high porosity substrate comprises an expanded polymer.

According to another example (“Example 29”) further to Example 28, wherein the expanded polymer is expanded polyurethane (ePU).

According to another example (“Example 30”) further to Example 22, the high porosity substrate is expanded polyparaxylylene (ePPX).

According to another example (“Example 31”) further to any one of Examples 22 to 30, the high porosity substrate is hydrophobic.

According to another example (“Example 32”) further to any one of Examples 22 to 31, the low porosity substrate and the high porosity substrate comprise are a same material.

According to another example (“Example 33”) further to any one of Examples 22 to 32, the cultivation substrate is a patterned substrate having a pattern of low porosity substrate and high porosity substrate.

According to another example (“Example 34”) further to Example 33, the pattern of low porosity substrate and high porosity substrate is an organized or selective pattern.

According to another example (“Example 35”) further to Example 33, the pattern of low porosity substrate and high porosity substrate is a random pattern.

According to another example (“Example 36”) further to any one of Examples 1-35, the cultivation system includes a nutrient phase associated with at least a portion of the cultivation substrate.

According to another example (“Example 37”) further to Example 36, the nutrient phase promotes growth of the seaweed and/or attachment of the seaweed to the cultivation substrate.

According to another example (“Example 38”) further to Example 36 or Example 37, at least a portion of the nutrient phase is entrained within the cultivation substrate, entrained on the cultivation substrate, or entrained within and on the cultivation substrate.

According to another example (“Example 39”) further to any one of Examples 36-38, the nutrient phase is present as a coating on a surface of the cultivation substrate.

According to another example (“Example 40”) further to any one of Examples 1 to 39, the cultivation substrate is provided by a plurality of particles in a dispersion formulated for deposition onto a backer layer or carrier substrate.

According to another example (“Example 41”) further to any one of Examples 1 to 40, the cultivation substrate is asymmetrical and includes the fibrillated submicron surface structure configured to retain seaweed only on one side.

According to another example (“Example 42”), a method for cultivating seaweed includes contacting a population of seaweed gametophytes and/or sporophytes with the cultivation substrate of the cultivation system of any one of Examples 1 to 41 until at least a portion of the population of seaweed gametophytes and/or sporophytes form a holdfast to the nanostructure of the cultivation substrate.

According to another example (“Example 43”) further to Example 42, the method includes positioning the cultivation system in an open-water environment after the portion of the population of seaweed gametophytes and/or sporophytes form a holdfast to the nanostructure of the cultivation substrate.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a photograph depicting a naturally occurring seaweed-bedrock interaction. Source: Morrison L, Feely M, Stengel DB, Blamey N, Dockery P, Sherlock A, Timmins É (2009) Seaweed attachment to bedrock: biophysical evidence for a new geophycology paradigm. Geobiology 7:477-487.

FIG. 1B is a detailed view of the area identified by the dashed-line box in FIG. 1A. Source: Morrison L, Feely M, Stengel DB, Blamey N, Dockery P, Sherlock A, Timmins É (2009) Seaweed attachment to bedrock: biophysical evidence for a new geophycology paradigm. Geobiology 7:477-487.

FIGS. 2A-2D are scanning electron microscopy (SEM) micrographs taken at various magnifications, depicting a nanostructure of a low porosity substrate in accordance with some embodiments. The scale bars provided in FIG. 2A, FIG. 2B, FIG. 2C, and FIG. 2D are 100 µm, 10 µm, 5 µm, and 5 µm, respectively.

FIGS. 3A-3D are scanning electron microscopy (SEM) micrographs taken at various magnifications, depicting the surface structure of a substrate in accordance with some embodiments. The scale bars provided in FIG. 3A, FIG. 3B, FIG. 3C, and FIG. 3D are 100 µm, 10 µm, 5 µm, and 5 µm, respectively.

FIG. 4 is a photograph depicting a kelp holdfast network on the surface of a low porosity substrate in accordance with some embodiments.

FIG. 5 is a collection of photographs depicting kelp growth on the membrane depicted in FIGS. 2A-D (two samples on left), and on the membrane depicted in FIGS. 3A-3D (two samples on right) in accordance with some embodiments.

FIG. 6 is a collection of photographs depicting nori growth on the membrane depicted in FIGS. 2A-D (two samples on left), and on the membrane depicted in FIGS. 3A-3D (two samples on right) in accordance with some embodiments.

FIG. 7 is a collection of photographs depicting dulse growth on the membrane depicted in FIGS. 2A-D (two samples on left), and on the membrane depicted in FIGS. 3A-3D (two samples on right) in accordance with some embodiments.

FIG. 8 is a scanning electron microscopy (SEM) micrograph depicting a microstructure of a high porosity substrate in accordance with some embodiments.

FIG. 9 is an SEM micrograph depicting the microstructure pictured in FIG. 1, but at a higher magnification.

FIG. 10 is an SEM micrograph depicting a microstructure of a high porosity substrate in accordance with some embodiments.

FIG. 11 is an SEM micrograph depicting the microstructure pictured in FIG. 3, but at a higher magnification.

FIG. 12 is a schematic illustration depicting a microstructure of a high porosity substrate in accordance with some embodiments.

FIG. 13 is the micrograph of FIG. 9 with cartoon representations of spores of either 10 µm or 30 µm in diameter overlaid thereon in inter-fibril spaces in accordance with some embodiments.

FIG. 14A is a cross-sectional SEM micrograph depicting ingrowth of dulse seaweed into a microstructure of a high porosity substrate in accordance with some em bodim ents.

FIG. 14B is a cross-sectional SEM micrograph depicting the ingrowth pictured in FIG. 14A, but at a higher magnification.

FIG. 14C is a cross-sectional optical fluorescence microscopy micrograph depicting ingrowth of dulse seaweed into a microstructure of a high porosity substrate in accordance with some embodiments.

FIG. 15 presents a surface SEM micrograph (top panel) depicting a microstructure of a high porosity substrate prior to seeding with sugar kelp spores in accordance with some embodiments, and an optical fluorescence microscopy micrograph (bottom panel) depicting the high porosity substrate following seeding with sugar kelp spores and germination thereof.

FIG. 16 presents two surface SEM micrographs taken at different magnifications depicting juvenile dulse ingrowth into a microstructure in accordance with some embodiments.

FIG. 17 is a surface optical fluorescence microscopy micrograph depicting ingrowth of dulse seaweed into a microstructure of a high porosity substrate in accordance with some embodiments.

Persons skilled in the art will readily appreciate the accompanying drawing figures referred to herein are not necessarily drawn to scale, but may be exaggerated or represented schematically to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to term inology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior / exterior direction.

### Description of Various Embodiments

The present disclosure relates to cultivation systems that include a cultivation substrate. The cultivation substrate is used for retention, culture, and/or growth of seaweed, and related methods and apparatuses. In some embodiments, the cultivation system is operable to grow seaweed in an open-water environment.

Cultivation systems according to the instant disclosure can be used in spore culture and growth, and spore and/or gametophyte/sporophyte transport and deposition. In certain embodiments, the cultivation substrates described herein can be used as an improved growth substrate for the growth and cultivation of seaweed forms (e.g., spores, gametophytes, sporophytes), resulting in improved yield and throughput relative to current cultivation practices

In some embodiments, the cultivation system includes a cultivation substrate which itself includes a low porosity substrate having a fibrillated submicron surface structure on at least one of the substrate’s surfaces. The fibrillated submicron surface structure of the low porosity substrate provides for the attachment of seaweed to the cultivation substrate through a seaweed holdfast.

A holdfast is a root-like structure at the base of seaweed that fastens it to a substrate such as a stone, for example. Holdfasts differ in shape and structure between species. Substrate type can also affect holdfast shape and structure. Having no nutrient absorbent function, serving only as an anchor, seaweed holdfasts differ from the roots of land plants.

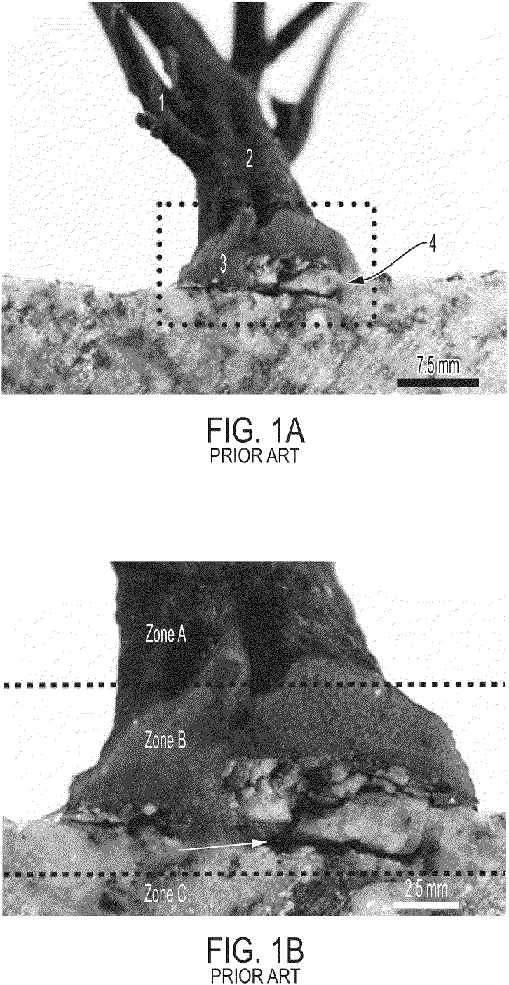

FIG. 1A depicts the zones of interaction between Fucus vesiculosus and granite bedrock. The cross section depicts the holdfast (arrow), and shows a seaweed side branch (1), main axis (2), holdfast region (3), and the holdfast-bedrock interface (4). FIG. 1B depicts a detailed view of the area within the dashed line box in FIG. 1A., detailing three zones of physicochemical activity comprising the holdfast interface. The arrow of FIG. 1B indicates rock fragments incorporated into and dispersed in the seaweed holdfast tissue.

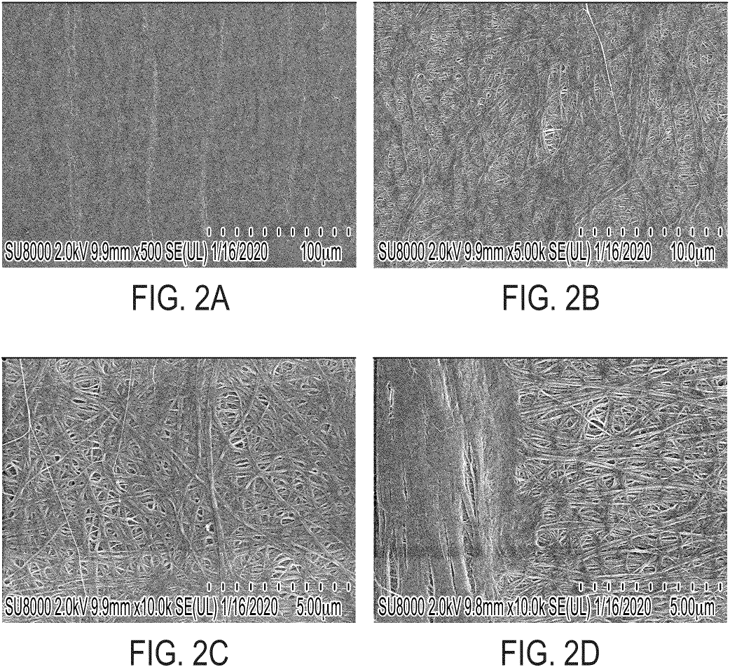

As described herein, it was surprisingly found that the nanostructure found on certain low porosity substrates would promote and support holdfast formation on the substrate’s surface. FIGS. 2A-2D are SEM micrographs depicting the nanostructure on the surface of a low porosity expanded polytetrafluoroethylene (ePTFE) substrate in accordance with some embodiments. FIGS. 2A-2C depict the nanostructure on a first side of the low porosity substrate at increasing magnifications. The presented scales are 100 µm (FIG. 2A), 10 µm (FIG. 2B), and 5 µm (FIG. 2C). FIG. 2D depicts the nanostructure on a second side of the low porosity substrate (provided scale of 5 µm). At the lowest magnification (FIG. 2A), the surface of the low porosity substrate appears to be nearly smooth. However, a fibrillated submicron surface structure is apparent at higher magnifications (FIGS. 2B-2D). As depicted, the fibrillated submicron surface structure is defined by a plurality of fibrils. The fibrils define inter-fibril spaces. In some embodiments, and as shown in FIGS. 2A-2D, the fibrils of the fibrillated submicron surface structure interconnect at nodes. In certain embodiments, the fibrillated submicron surface structure is free of nodes, or substantially free of nodes.

The fibrils have a defined average inter-fibril distance, which in some embodiments may be from about 1 nm to about 1000 nm, from about 1 nm to about 500 nm, from about 1 nm to about 200 nm, from about 1 nm to about 50 nm, from about 1 nm to about 20 nm, from about 1 nm to about 10 nm, from about 1 nm to about 5 nm, from about 5 nm to about 500 nm, from about 5 nm to about 200 nm, from about 5 nm to about 100 nm, from about 5 nm to about 50 nm, from about 5 nm to about 20 nm, from about 5 nm to about 10 nm, from about 10 nm to about 100 nm, from about 10 nm to about 500 nm, from about 10 nm to about 200 nm, from about 10 nm to 100 nm, from about 10 nm to about 75 nm, from about 10 nm to about 50 nm, from about 10 nm to about 25 nm, from about 25 nm to about 200 nm, from about 25 nm to about 150 nm, from about 25 nm to about 100 nm, from about 25 nm to about 50, from about 50 nm to about 200 nm, from about 50 nm to about 150 nm, from about 50 nm to about 100 nm, from about 100 nm to about 500 nm, from about 100 nm to about 200 nm, from about 100 nm to about 150 nm, from about 150 nm to about 500 nm, or from about 150 nm to about 200 nm. In some embodiments, the fibrils may have an average inter-fibril distance of about 1 nm, about 2 nm, about 3 nm, about 4 nm, about 5 nm, about 10 nm, about 20 nm, about 30 nm, about 40 nm, about 50 nm, about 60 nm, about 70 nm, about 80 nm, about 90 nm, about 100 nm, about 110, about 120 nm, about 130 nm, about 140 nm, about 150 nm, about 160 nm, about 170 nm, about 180 nm, about 190 nm, about 200 nm, about 250 nm, about 300 nm, about 400 nm, about 500 nm, about 600 nm, about 700 nm, about 800 nm, about 900 nm, or about 1000 nm.

In certain embodiments, the fibrillated submicron surface structure has an average depth of about 1000 nm or less. That is, the fibrillated submicron surface structure exists on the surface of the low porosity substrate up to a depth of about 1000 nm or less into the low porosity substrate in the z dimension. In some embodiments, the average depth of the fibrillated submicron surface structure may be from about 1 nm to about 1000 nm, from about 1 nm to about 500 nm, from about 1 nm to about 300 nm, from about 1 nm to about 100 nm, from about 1 nm to about 50 nm, from about 1 nm to about 20 nm, from about 1 nm to about 10 nm, from about 1 nm to about 5 nm, from about 5 nm to about 1000 nm, from about 5 nm to about 500 nm, from about 5 nm to about 300 nm, from about 5 nm to about 100 nm, from about 5 nm to about 50 nm, from about 5 nm to about 20 nm, from about 5 nm to about 10 nm, from about 10 nm to about 1000 nm, from about 10 nm to about 500 nm, from about 10 nm to about 300 nm, from about 10 nm to about 100 nm, from about 10 nm to about 75 nm, from about 10 nm to about 50 nm, from about 10 nm to about 25 nm, from about 25 nm to about 1000 nm, from about 25 nm to about 500 nm, from about 25 nm to about 300 nm, from about 25 nm to about 100 nm, from about 25 nm to about 75 nm, from about 25 nm to about 50 nm, from about 50 nm to about 1000 nm, from about 50 nm to about 500 nm, from about 50 nm to about 300 nm, or from about 50 nm to about 100 nm. In some embodiments, the fibrillated submicron surface structure may have an average depth of about 1 nm, about 2 nm, about 3 nm, about 4 nm, about 5 nm, about 10 nm, about 20 nm, about 30 nm, about 40 nm, about 50 nm, about 60 nm, about 70 nm, about 80 nm, about 90 nm, or about 100 nm.

In some embodiments, the fibrillated submicron surface structure includes two or more layers of nodes and fibrils. Such an arrangement is depicted, for example, in FIG. 2C, where fibrils appear to overlap with others. In certain embodiments, the depth of the fibrillated submicron surface structure is indicative of the layered nod and fibril configuration.

In some embodiments, the low porosity substrate has a porosity of about 10% or less. The porosity of the low porosity substrate may be from about 1% to about 10%, from about 1% to about 5%, or from about 5% to about 10%. In some embodiments, the porosity of the low porosity substrate by be about 1%, about 2%, about 3%, about 4%, about 5%, about 6%, about 7%, about 8%, about 9%, or about 10%.

The low porosity substrate can have a thickness of about 1 mil (0.001 inch; 25.4 µm) to about 30 mil (0.03 inch; 254 µm). In some embodiments, the thickness of the low porosity substrate may be from about 1 mil (0.001 inch; 25.4 µm) to about 10 mil (0.01 inch; 254 µm), or from about 1 mil (0.001 inch; 25.4 µm) to about 5 mil (0.005 inch; 254 µm). In some embodiments, the thickness of the low porosity substrate is about 1 mil (0.001 inch; 25.4 µm), about 2 mil (0.002 inch; 50.8 µm), about 3 mil (0.003 inch; 76.2 µm), about 4 mil (0.004 inch; 101.6 µm), about 5 mil (0.005 inch; 127 µm), about 6 mil (0.006 inch; 152.4 µm), about 7 mil (0.007 inch; 177.8 µm), about 8 mil (0.008 inch; 203.2 µm), about 9 mil (0.009 inch; 228.6 µm), about 10 mil (0.01 inch; 254 µm), about 12 mil (0.012 inch; 304.8 µm), about 15 mil (0.015 inch; 381 µm), about 20 mil (0.02 inch; 508 µm), about 25 mil (0.025 inch; 635 µm), or about 30 mil (0.03 inch; 762 µm).

The fibrillated submicron surface structure of the low porosity substrate of the cultivation substrate is configured to retain seaweed by a holdfast. The depth of the fibrillated submicron surface structure and porosity of the low porosity substrate are sufficient to retain seaweed by a holdfast, while resisting ingrowth of seaweed into the low porosity substrate past the depth of the node and fibril nanostructure. In some embodiments, a plurality of seaweed spores are seeded onto the cultivation substrate and allowed to develop into juvenile seedling, during which time the seaweed develops holdfast structures on the surface of the cultivation substrate. In other embodiments, a plurality of juvenile seedlings (e.g., sporophytes and/or gametophytes) are directly seeded onto the cultivation substrate and allowed to form holdfasts thereon. The plurality of seaweed spores and/or juvenile seaweed may all be of the same species, or of two or more different species. In some embodiments, two different seaweed species display a symbiotic relationship when cultured or grown together.

In addition to retaining seaweed via a seaweed holdfast, cultivation systems and substrates of the instant disclosure can promote germination of and growth of seeded seaweed spores, and growth of juvenile and mature seaweed. The cultivation substrate can, for example, create a microenvironment conducive to the germination of and growth from the seeded seaweed spores, and growth of juvenile and mature seaweed.

In certain embodiments, the cultivation substrate provides a selective nanostructure conducive to the formation of holdfasts and subsequent growth of one or more target seaweed species while inhibiting or preventing attachment or growth of non-target species or other organisms. That is, the nanostructure of the cultivation substrate supports attachment and growth of seaweed species while inhibiting biofouling. In some embodiments, where biofouling species (e.g., non-target species or other organisms) do attach to the cultivation substrate, the attachment is weaker than that of the target seaweed species, and the biofouling species are removable by, for example, rinsing. In such embodiments, the physical removal of the biofouling species does not result in a significant dislodgement of the target species.

In some embodiments, the cultivation substrates encourage quick and healthy growth of target species, allowing the target species to produce and secrete natural anti-fouling compounds before biofouling species are able to establish on the cultivation substrate. The target species thus, in addition to the fibrillated submicron surface structure of the low porosity substrate, contributes to anti-biofouling.

A selective nanostructure can be achieved by, for example, providing a combination of inter-fibril distance, substrate porosity, and depth of the fibrillated submicron surface structure that supports attachment and growth of the target seaweed species while inhibiting or preventing attachment and growth of biofouling species.

Good settlement and attachment are critical for the successful cultivation of a seaweed crop; juvenile plants must be attached firmly enough so as not to be separated from the cultivation substrate in the extreme exposure of the open ocean. All juvenile seaweed are prone to inhibition from biofouling, which is most often caused by an overgrowth of other algal species, such as diatoms, filamentous brown algae, and green algae. Biofouling issues are most prevalent at the farm site when first set out, and small enough to be in danger of smothering, although biofouling can sometimes occur in the nursery during seed production. An ideal cultivation substrate would provide for secure attachment of target species, while discouraging growth of biofouling organisms.

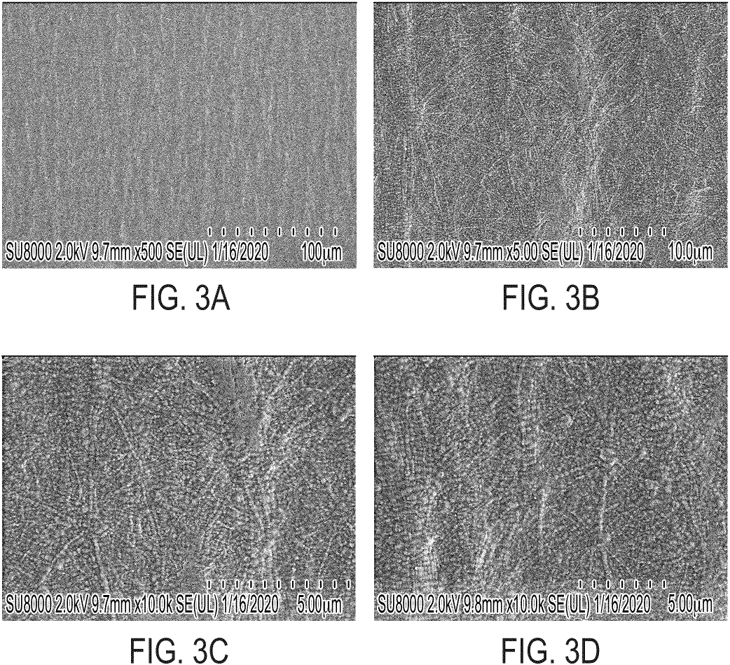

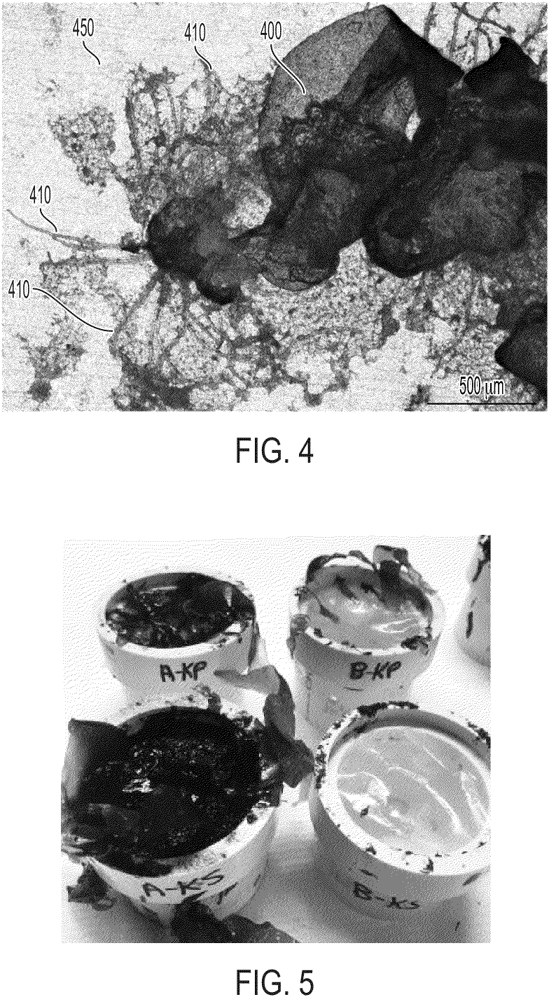

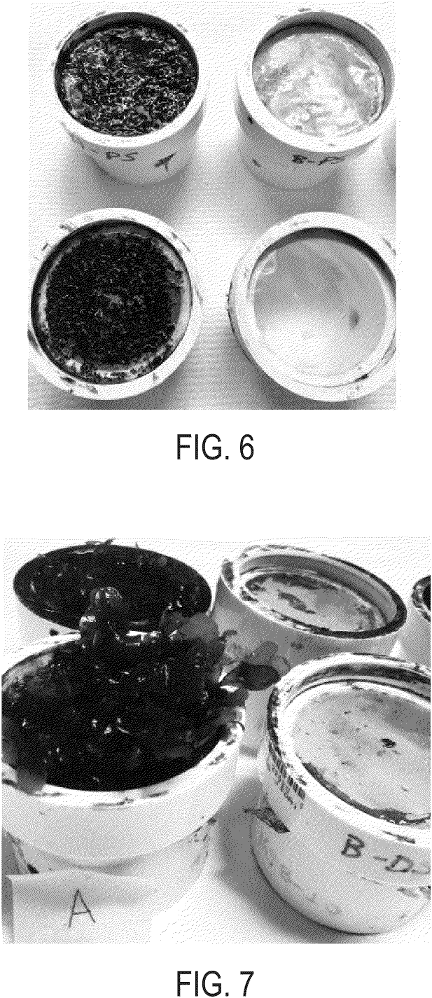

The fibrillated submicron surface structure of the low porosity substrates described herein support such strong attachment and growth of seaweed, while inhibiting biofouling. FIG. 4 depicts a juvenile kelp plant 400 attached to a low porosity substrate 450 having a fibrillated submicron surface structure. Juvenile kelp plant 400 is attached to the low porosity substrate 450 via a holdfast 410, which appears as a network of projections emitting from the base of the juvenile kelp plant 400. As depicted in the two samples on the left of each of FIGS. 5, 6, and 7, such attachment was observed when seeding and growing kelp, nori, and dulse, respectively, on a low porosity substrate having a fibrillated submicron surface structure according to some embodiments. Conversely, a low porosity substrate lacking the fibrillated submicron surface structure failed to retain seaweed, as depicted in the two samples on the right of each of FIGS. 5, 6, and 7. The surface structure of the low porosity substrate lacking a fibrillated submicron surface structure is depicted by FIGS. 3A-3D. FIGS. 3A-3D are SEM micrographs depicting the surface structure of a low porosity ePTFE substrate lacking a surface node and fibril nanostructure. FIGS. 3A-3C depict the surface structure on a first side of the low porosity substrate at increasing magnifications. The presented scales are 100 µm (FIG. 3A), 10 µm (FIG. 3B), and 5 µm (FIG. 3C). FIG. 3D depicts the surface structure on a second side of the low porosity substrate (provided scale of 5 µm). At the lowest magnification (FIG. 3A), the surface of the low porosity substrate appears to be nearly smooth, and similar to that of the low porosity substrate depicted in FIG. 2A. At higher magnification, it is clear that the substrate lacks a fibrillated submicron surface structure (FIGS. 3B-3D).

In certain embodiments, in addition to the low porosity substrate, the cultivation substrate includes a high porosity substrate. Certain embodiments, the high porosity substrate has a porosity of at least 30%, and a node and fibril microstructure characterized by an average inter-fibril distance from about 1 µm to about 500 µm, or average pore size of about 1 µm to about 500 µm. The high porosity substrate can function, for example, to intersperse the low porosity substrate, help control seaweed localization on the low porosity substrate, and function to deliver nutrients to growing seaweed. In some embodiments, the high porosity substrate can retain and support growth of spores (e.g. retain and support growth of algal spores and mature seaweed therefrom), and/or inhibit or prevent retention of spores and/or biofouling organisms. Whether the high-porosity retains and supports spore growth or inhibits such retention depends on the characteristics of the high porosity substrate’s characteristics, such as porosity and inter-fibril distance.

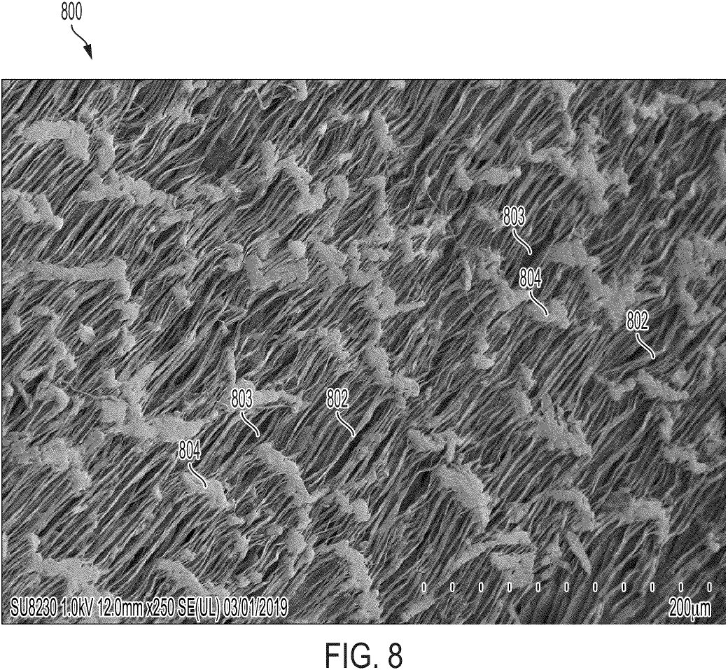

In some embodiments, the high porosity substrate has a microstructure including a plurality of fibrils defining an average inter-fibril distance. FIG. 8 is an SEM micrograph depicting a microstructure 100 of high porosity substrate including a fibrillated material according to some embodiments. The fibrillated material depicted in FIG. 1 having the microstructure 800 is expanded polytetrafluoroethylene (ePTFE). As depicted, the microstructure 800 is defined by a plurality of fibrils 802 that interconnect nodes 804. The fibrils 802 define inter-fibril spaces 803.

The fibrils 803 have a defined average inter-fibril distance, which in some embodiments may be from about 1 µm to about 500 µm,1 µm to about 200 µm, from about 1 µm to about 50 µm, from about 1 µm to about 20 µm, from about 1 µm to about 10 µm, from about 1 µm to about 5 µm, from about 5 µm to about 50 µm, from about 5 µm to about 20 µm, from about 5 µm to about 10 µm, from about 10 µm to about 100 µm, from about 10 µm to about 75 µm, from about 10 µm to about 50 µm, from about 10 µm to about 25 µm, from about 25 µm to about 200 µm, from about 25 µm to about 150 µm, from about 25 µm to about 100 µm, from about 25 µm to about 50, from about 50 µm to about 200 µm, from about 50 µm to about 150 µm, from about 50 µm to about 100 µm, from about 100 µm to about 200 µm, from about 100 µm to about 150 µm, from about 150 µm to about 200 µm, or from about 200 µm to about 500 µm. In some embodiments, the fibrils 802 may have an average inter-fibril distance of about 1 µm, about 2 µm, about 3 µm, about 4 µm, about 5 µm, about 10 µm, about 20 µm, about 30 µm, about 40 µm, about 50 µm, about 60 µm, about 70 µm, about 80 µm, about 90 µm, about 100 µm, about 110, about 120 µm, about 130 µm, about 140 µm, about 150 µm, about 160 µm, about 170 µm, about 180 µm, about 190 µm, about 200 µm, about 300 µm, about 400 µm, or about 500 µm.

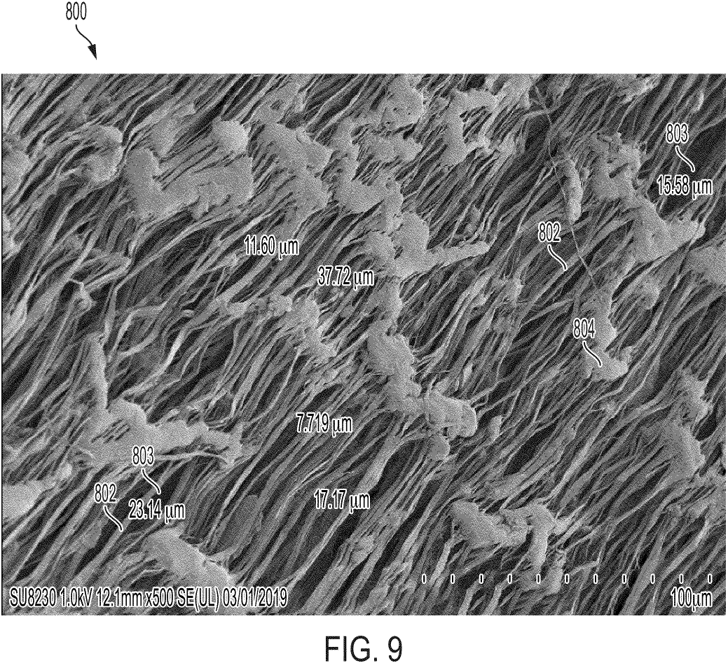

FIG. 9 is a higher magnification SEM micrograph of the microstructure depicted in FIG. 8. FIG. 9 identifies the dimension of select inter-fibril spaces 803 in µm.

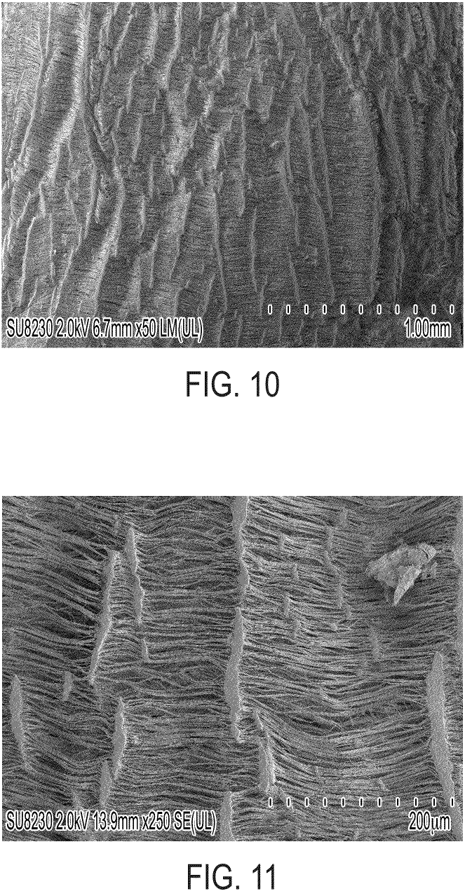

FIG. 10 is an SEM micrograph depicting another microstructure of a high porosity substrate that includes a fibrillated ePTFE material according to some em bodim ents.

FIG. 11 is a higher magnification SEM micrograph of the microstructure depicted in FIG. 10.

In some embodiments, at least some of the fibrils 802 are sufficiently spaced from each other to retain a spore in an inter-fibril space 802. In other embodiments, the fibrils 802 are sufficiently spaced from each other to inhibit or prevent retention of a spore in an inter-fibril space 802.

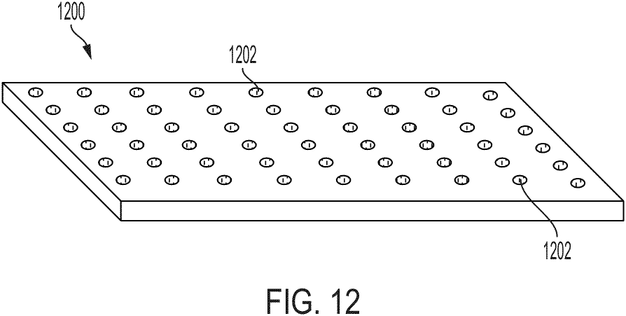

FIG. 12 is a perspective view of a schematic representation of the microstructure of a cultivation substrate according to some embodiments. As depicted, the microstructure 1200 is defined by a plurality of pores 1202.

The pores 1202 may be round, approximately round, or oblong. The pores 1202 may have a diameter or approximate diameter from about 1 µm to about 500 µm, 1 µm to about 200 µm, from about 1 µm to about 50 µm, from about 1 µm to about 20 µm, from about 1 µm to about 10 µm, from about 1 µm to about 5 µm, from about 5 µm to about 50 µm, from about 5 µm to about 20 µm, from about 5 µm to about 10 µm, from about 10 µm to about 100 µm, from about 10 µm to about 75 µm, from about 10 µm to about 50 µm, from about 10 µm to about 25 µm, from about 25 µm to about 200 µm, from about 25 µm to about 150 µm, from about 25 µm to about 100 µm, from about 25 µm to about 50, from about 50 µm to about 200 µm, from about 50 µm to about 150 µm, from about 50 µm to about 100 µm, from about 100 µm to about 200 µm, from about 100 µm to about 150 µm, from about 150 µm to about 200 µm, or from about 200 µm to about 500 µm. In some embodiments, the pores 1202 may have a diameter or approximate diameter of about 1 µm, about 2 µm, about 3 µm, about 4 µm, about 5 µm, about 10 µm, about 20 µm, about 30 µm, about 40 µm, about 50 µm, about 60 µm, about 70 µm, about 80 µm, about 90 µm, about 100 µm, about 110, about 120 µm, about 130 µm, about 140 µm, about 150 µm, about 160 µm, about 170 µm, about 180 µm, about 190 µm, about 200 µm, about 300 µm, about 400 µm, or about 500 µm

In some embodiments, the inter-fibril spaces 803 of FIG. 8 form the pores 1202 of FIG. 12. That is, a microstructure 800 having a plurality of fibrils 802 may form the porous microstructure 1200. However, not all microstructures 1200 having pores 1202 are fibrillated.

In some embodiments, the microstructure of the high porosity substrate is configured to retain spores and sporophytes, gametophytes, or other organisms grown from the retained spores. In some embodiments, the microstructure is configured to retain algal spores, algal sporophytes and/or gametophytes, plant spores, seedlings, bacterial endospores, fungal spores, or a combination thereof. In some embodiments, the cultivation substrate retains a plurality of spores and/or organisms grown therefrom (e.g., sporophytes and/or gametophytes). The plurality of spores and/or organisms may all be of the same type, or of two or more different types. In some embodiments, the high porosity substrate retains seaweed spores and/or seaweed of the same type that is seeded on and attached to the low porosity substrate. In other embodiments, the high porosity substrate retains seaweed spores and/or seaweed of a different type than what is seeded on and attached to the low porosity substrate. In some embodiments, the cultivation substrate retains two different spore types that display a symbiotic relationship when cultured or grown together. For sake of simplicity, throughout this disclosure reference will be made to “spores” in relation to the low porosity substrate, although gametophytes, sporophytes, seedlings, or other organisms grown from the spores are also contemplated by this term and are considered to be within the purview of the disclosure.

In some embodiments, in addition to retaining spores, high porosity substrates promote germination of and growth from the retained spores. That is, the high porosity substrates viably maintain the retained spores. In certain embodiments, the microstructure is configured to irremovably anchor at least a portion of a spore.

The high porosity substrate, for example, creates a microenvironment conducive to the germination of and growth from the retained spores. In some embodiments, the microstructure is initially in a first retention phase, where the microstructure functions to retain and maintain a target spore. The microstructure subsequently is in a second growth phase, where germination of the spore is induced, and ingrowth of sporelings (e.g., sporophytes, gametophytes, seedlings, etc.) from the spore on and/or into the microstructure, thereby resulting in a mechanical coupling, or anchoring, of the sporelings to the microstructure. Thus, in some embodiments, the microstructure is configured to irremovably anchor germinated spores, preventing loss of the germinated spores during, for example, transport or placement in the field (e.g., an open-water environment), or loss to environmental factors (e.g., currents).

In certain embodiments, the high porosity substrate creates a selective microenvironment conducive to the germination of and growth from a target spore while inhibiting or preventing germination, growth, and/or proliferation of non-target spores or other cells. A selective microenvironment can be achieved by, for example, providing a combination of inter-fibril distance and/or pore size, material density, ratio of inter-fibril distance to average density of material, depth or thickness, hydrophobicity, and presence or absence of nutrient sources, moisture, bioactive agents, and adhesives that supports germination of and growth from the target spore while inhibiting or preventing germination, growth, and/or proliferation of non-target spores or other cells.

Several factors may affect retention and/or viable maintenance of the spores and organisms grown therefrom. Such factors include, for example, the inter-fibril distance and/or pore size, material density, a ratio of inter-fibril distance to average density of material, depth or thickness, hydrophobicity, and presence or absence of nutrient sources, moisture, bioactive agents, and adhesives. These factors will each be described in more detail.

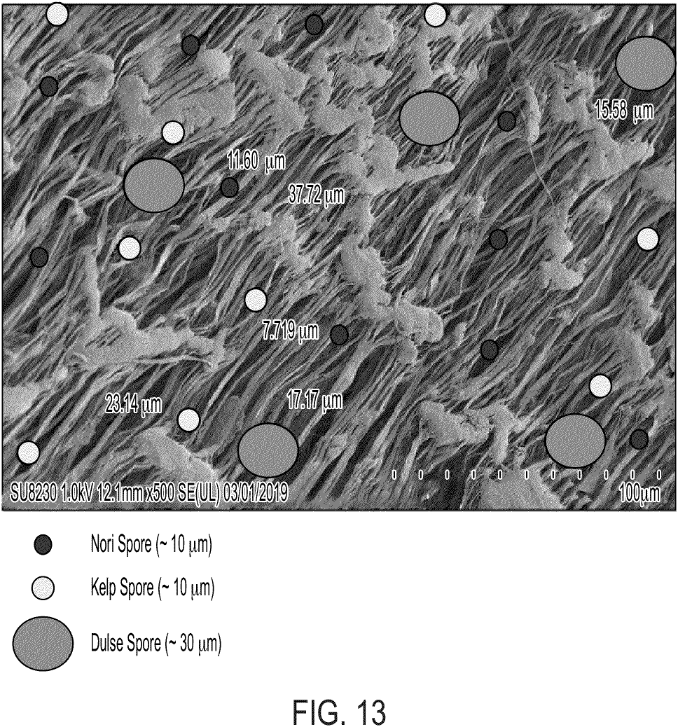

The distance between two fibrils (i.e., inter-fibril distance) defines an inter-fibril space 803. In some embodiments, an inter-fibril space 803 - and thus the inter-fibril distance - is sufficient to retain a spore therein; the spore is retained between the two fibrils defining the inter-fibril space. The inter-fibril distance is sufficient to allow at least a portion of the spore to enter between the two fibrils defining the inter-fibril space 803. In some embodiments, the spore is thereby retained within the microstructure of the cultivation substrate. FIG. 13 is a modified version of the photograph of FIG. 9, depicting a microstructure of a high porosity substrate including a fibrillated material and overlaid with representative spores having a diameter of either about 10 µm (e.g., nori and kelp spores) or about 30 µm (e.g., dulse spores). FIG. 13 illustrates how and where target spores may enter between the two fibrils defining an inter-fibril space.

In some embodiments, the average inter-fibril distance of the high porosity substrate is controlled in order to encourage ingress of at least portions of spores into the microstructure. For exam ple, where it is desirous for the microstructure to retain spores of dulse (Palmaria palmata), which have a diameter of about 30 µm, the average inter-fibril distance of the high porosity substrate microstructure is about 30 µm, or slightly larger (e.g., about 32 µm to about 35 µm). Where it is desirous for the high porosity microstructure to retain spores of nori or kelp, which each have a spore having a diameter of about 10 µm, the average inter-fibril distance of the microstructure is about 10 µm, or slightly larger (e.g., about 12 µm to about 15 µm). In some embodiments, it may be desirous to retain spores of multiple species (e.g., dulse, nori, and kelp). In such embodiments, the average inter-fibril distance is sufficient to allow at least a portion of the spores of the multiple species to enter the inter-fibril space and be retained there. In some embodiments, target spores have a diameter of about 0.5 µm to about 200 µm.

In some embodiments, about half of the target spore may enter the inter-fibril space 803 in the high porosity substrate. In such embodiments, the inter-fibril distance is at least equal to a dimension (e.g., diameter or width) of the target spore. In some embodiments, the inter-fibril distance is slightly larger than the dimension of the target spore. This allows for the entire spore to enter the inter-fibril space 803 and be retained therein.

In some embodiments, more than half of the target spore may enter the inter-fibril space 803 of the high porosity substrate, up to the entire spore. In such embodiments, the portion of the spore entering the inter-fibril space 803 may be governed by the depth of a pore, the opening of which is defined by the inter-fibril space. The depth of the pore may be controlled by, for example, material density.

In some embodiments, only a portion of the spore enters the inter-fibril space 803 of the high porosity substrate. Therefore, in instances where the inter-fibril distance is less than the diameter of the target spore, the target spore may only partially enter the inter-fibril space 803. Where the target spore only partially enters the inter-fibril space 803, the target spore may none-the-less be retained therein if a sufficient portion of the target spore enters the inter-fibril space 803. In some embodiments, a substance such as an adhesive applied to the microstructure may reduce the portion of the spore required to enter the inter-fibril space 803 and aid in retention.

In some embodiments, the microstructure of the high porosity substrate is formed by a non-fibrillated material. In certain embodiments, the pore openings 1202 are inherent to the material of the cultivation substrate. It will be recognized that different materials may have different pore opening properties, and that a material may be manufactured or otherwise manipulated to provide the desired pore opening properties. In other embodiments, the pore openings 1202 are formed by micro drilling techniques such as, for example: mechanical micro drilling, such as ultrasonic drilling, powder blasting or abrasive water jet machining (AWJM); thermal micro drilling, such as laser machining; chemical micro drilling, including wet etching, deep reactive ion etching (DRIE) or plasma etching; and hybrid micro drilling techniques, such as spark-assisted chemical engraving (SACE), vibration-assisted micromachining, laser-induced plasma micromachining (LIPMM), and water-assisted micromachining.

In those embodiments where the microstructure of the high porosity substrate is formed by a non-fibrillated material, the pore openings 1202 act much like the inter-fibril spaces 103 described and are of a sufficient size to allow at least a portion of a target spore to enter the pore opening 1202. In some embodiments, the spore is thereby retained within the microstructure of the cultivation substrate. In some embodiments, the size of pore openings 1202 is controlled to encourage ingress of a least portions of target spores into the microstructure. For example, where it is desirous for the microstructure of the high porosity substrate to retain spores of dulse (Palmaria palmata), which have a diameter of about 30 µm, the pore openings 1202 of the microstructure have a diameter of about 30 µm, or slightly larger (e.g., about 32 µm to about 35 µm). In some embodiments, target spores have a diameter of about 0.5 µm to about 200 µm.

In some embodiments, about half of the target spore may enter the pore opening 1202 in the high porosity substrate. In such embodiments, the pore opening is at least equal to a dimension (e.g., diameter or width) of the target spore. In some embodiments, the pore opening is slightly larger than the dimension of the target spore. This allows for the entire spore to enter the pore opening 1202 and be retained therein.

In some embodiments, more than half of the target spore may enter the pore opening 1202 in the high porosity substrate, up to the entire spore. In such embodiments, the portion of the spore entering the pore opening 1202 may be governed by the pore depth. The depth of the pore may be controlled by, for example, material density.

In some embodiments, only a portion of the spore enters the pore opening 1202. Therefore, where the pore opening is smaller than the diameter of the target spore, the target spore may only partially enter the pore opening 1202. Where the target spore only partially enters the pore opening 1202, the target spore may none-the-less be retained therein when a sufficient portion of the target spore enters the pore opening. In some embodiments, a substance such as an adhesive applied to the microstructure may reduce the portion of the spore required to enter the pore opening 1202 and aid in retention.

In some embodiments, the high porosity substrate is a low-density material. The low-density material may be fibrillated or non-fibrillated, and in some embodiments, defines the microstructure of the cultivation substrate. The density of the low-density material may be about 0.1 g/cm³, about 0.2 g/cm³, about 0.3 g/cm³, about 0.4 g/cm³, about 0.5 g/cm³, about 0.6 g/cm³, about 0.7 g/cm³, about 0.8 g/cm³, about 0.9 g/cm³, or about 1.0 g/cm³. In some embodiments, the density of the low-density material is from about 0.1 g/cm³ to about 1 g/cm³.

In some embodiments, the low-density material provides a sufficient pore depth to retain spores in inter-fibril spaces 803 or pore openings 1202.

In some embodiments, the dimensions of the pore openings (length (µm) and width (µm)), whether formed by a fibrillated or non-fibrillated material, together with the depth at which target spores enter the pores (µm) define a capture ratio. Each spore type may have a different capture ratio required for adequate retention of spores by the microstructure of the high porosity substrate. The required capture ratio may be influenced by the properties of the material making up the microstructure of the high porosity substrate and the presence or absence of nutrients, adhesives, and/or bioactive agents.

In some embodiments, the low-density material allows the spore to germinate and grow into the low-density material. For example, as dulse spores retained in a low-density material having a microstructure described herein develop into gametophytes and then sporophytes, the dulse grows into the low-density material in all three dimensions (i.e., horizontally in x- and y-dimensions and depth-wise in the z-dimension). This three-dimensional growth allows for improved retention of the dulse gametophytes and sporophytes.

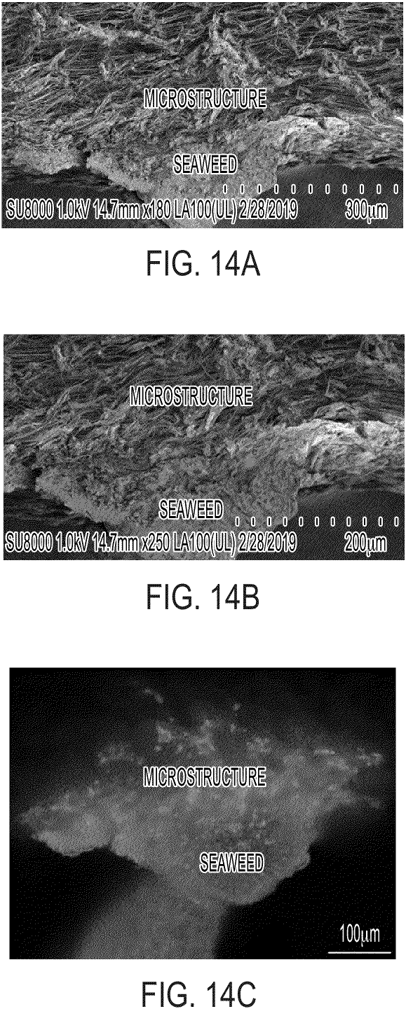

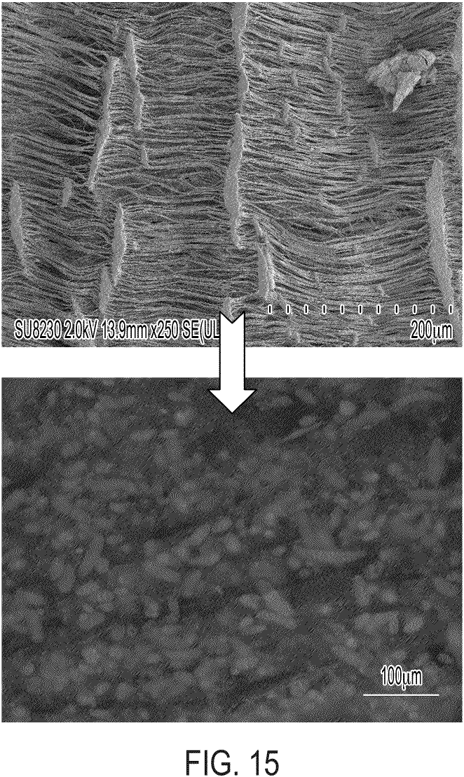

FIGS. 14A and 14B are cross-sectional SEM micrographs taken at two different magnifications of a low-density, high porosity microstructured material according to some embodiments, depicting dulse seaweed three-dimensional ingrowth into the low-density material. FIG. 14C is a cross-sectional micrograph generated using optical fluorescence microscopy depicting dulse seaweed ingrowth into the low-density material.

FIG. 15 (top panel) is an SEM micrograph of the surface of a low density, high porosity microstructured material according to some embodiments. FIG. 15 (bottom panel) depicts the same cultivation substrate material as the top panel following seeding with sugar kelp spores and germination thereof.

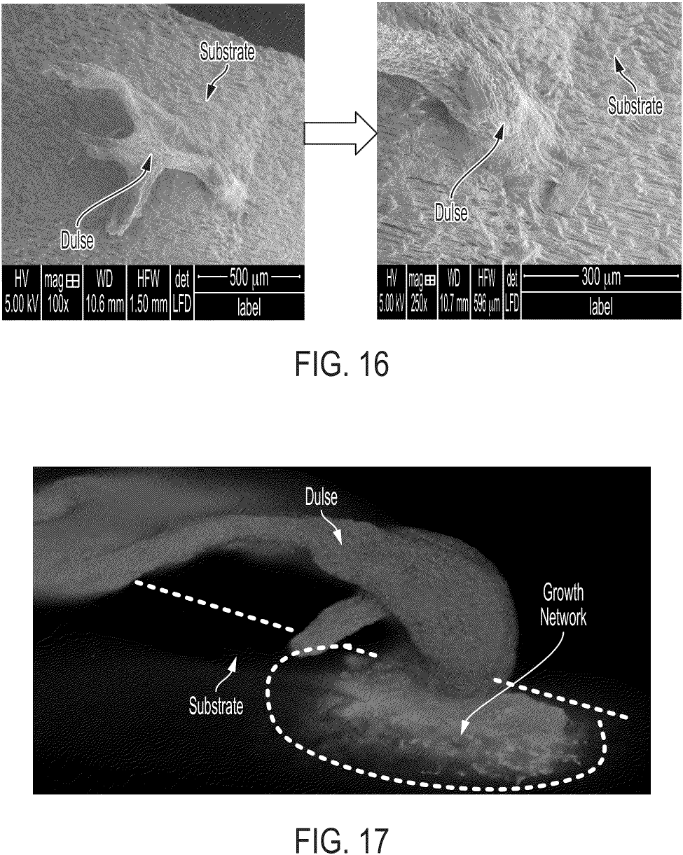

FIG. 16 depicts SEM micrographs of the surface of a microstructure taken at two different magnifications, where dulse seaweed can clearly be seen to be attached to and growing into the microstructure. FIG. 17 depicts a fluorescence microscopy micrograph of the surface of a microstructure to which the dulse seaweed is attached and growing into the microstructure. The seaweed growth is observed to be growing into the microstructure in a ‘growth network’ in all three dimensions.

It is evident from the micrographs of FIG. 14A - FIG. 17 that the dulse seaweed is able to grow into the microstructure of the fibrillated high porosity ePTFE substrate in all three dimensions, securely anchoring the seaweed within the microstructure.

In some embodiments, germinated spores grow deep into the microstructure of the high porosity substrate. This deep ingrowth and incorporation into the microstructure gives additional benefits in protecting the germinated spores from external environments (e.g., in the case of seaweed gametophytes, the sea and its currents). In some embodiments, the depth of penetration of the germinated spores relative to the initial size of the spore is from about 1:1 to about 200:1. For example, for a dulse spore having an initial diameter of about 30 µm, the dulse sporophyte may grow into the microstructure to a depth of about 30 µm to about 6 mm.

In some embodiments, the low-density, high porosity material has a thickness sufficient to allow for a desired level of ingrowth. In some embodiments, the cultivation substrate includes a single layer of the low-density material. In some embodiments, the cultivation substrate includes two or more layers of the low-density material. In certain embodiments, the two or more layers are present in a laminate, i.e., a laminate of a plurality of layers of the low-density material.

In some embodiments, the inter-fibril distance and the density of the high porosity material having a microstructure defines a ratio of the average inter-fibril distance (µm) to the average density (g/cm³) of the fibrillated material. In some embodiments, the ratio of the average inter-fibril distance (µm) to the average density (g/cm³) of the fibrillated material may be about 1:1, about 10:1, about 20:1, about 30:1, about 40:1, about 50:1, about 60:1, about 70:1, about 80:1, about 90:1, about 100:1, about 125:1, about 150:1, about 175:1, about 200:1, about 225:1, about 250:1, about 275:1, about 300:1, about 325:1, about 350:1, about 375:1, about 400:1, about 425:1, about 450:1, about 475:1, about 500:1, about 550:1, about 600:1, about 650:1, about 700:1, about 750:1, about 800:1, about 900:1, about 1000:1, about 1250:1, about 1500:1, about 1750:1, or about 2000:1. In some embodiments, the ratio of the average inter-fibril distance (µm) to the average density (g/cm³) of the fibrillated material is from about 1:1 to about 2000:1.

In some embodiments, the cultivation substrate (i.e., the low porosity substrate, the high porosity substrate, or both) includes one or more adhesives. An adhesive may be applied to the surface of the fibrillated submicron surface structure or the of the low porosity substrate or the microstructure of the high porosity substrate, imbibed within the low porosity substrate or the high porosity substrate, or both applied to the surface of the fibrillated submicron surface structure or the of the low porosity substrate or the microstructure of the high porosity substrate and im bibed within the low porosity substrate or the high porosity substrate. In some embodiments, the adhesive includes one or more cell-adhesive ligands specific to the spore(s) to be retained by the cultivation substrate.

In some embodiments, a cultivation substrate described herein (i.e., the low porosity substrate, the high porosity substrate, or both) includes a nutrient phase associated with at least a portion of the cultivation substrate. The nutrient phase serves to viably maintain spores, germinated spores retained by the cultivation substrate, and growing organisms (e.g., juvenile seaweed). In some embodiments, the nutrient phase promotes germination of and growth from retained spores within the microstructure of the high porosity substrate. In some embodiments, the nutrient phase acts to maintain and/or encourage attachment to the low porosity substrate and/or the high porosity substrate, or maintain and/or encourage ingrowth into or integration within the microstructure of the high porosity substrate.

In some em bodim ents, the nutrient phase acts as a chem oattractant capable of attracting the spores or juvenile organisms (e.g., seaweed sporophytes and/or gametophytes) to predetermined locations of the cultivation substrate to which the nutrient phase is applied or included.

The nutrient phase can be included as a filler in the low porosity substrate, on the fibrillated submicron surface structure of the low porosity substrate, within the microstructure of the high porosity substrate, on the microstructure (e.g., on its surface) of the high porosity substrate, or any combination thereof. In some embodiments, the nutrient phase is applied to a surface of the cultivation substrate as a coating. In some embodiments, the nutrient phase is included within one or more materials forming the cultivation substrate. Where the nutrient phase in included within a material forming the low porosity substrate, the nutrient phase may encourage attachment and holdstrong development. Where the nutrient phase is included within a material forming the high porosity substrate, the nutrient phase may encourage ingrowth into or integration within the microstructure. By encouraging growth of seaweed, the nutrient phase can assist in preventing biofouling, as healthy, quick-growing seaweed are known to produce and release their own natural antifouling compounds.

In some embodiments, the nutrient phase includes at least one nutrient beneficial to the target seaweed species and/or target spore and resulting germinated spore to be attached to or retained by the cultivation substrate. For example, where dulse seaweed is to attached to the fibrillated submicron surface structure of the low porosity substrate or retained by the microstructure of the high porosity substrate, the nutrient phase can include macronutrients (e.g., nitrogen, phosphorous, carbon, etc.), micronutrients (e.g., iron, zinc, copper, manganese, molybdenum, etc.), and vitamins (e.g., vitamin B₁₂, thiamine, biotin) that will support the growth and health of the germinated dulse spore. The nutrients of the nutrient phase can be provided in various forms. For example, nitrogen can be provided as ammonium nitrate (NH₄NO₃), ammonium sulfate ((NH₄)₂SO₄), calcium nitrate (Ca(NO₃)₂), potassium nitrate (KNO₃), urea (CO(NH₂)₂), etc. It will be recognized by those of skill in the art which nutrients would be beneficial to include in the nutrient phase so as to viably maintain the spores and resulting germinated spores to be retained by the cultivation substrate.

Which nutrients to include in the nutrient phase will depend on which spores are to be retained by the cultivation substrate, as various spore types, germinated spores, and growing organisms (e.g., seaweed) will have different nutrient needs. Nutrient selection may also depend on the intended use of the cultivation system. For example, where a cultivation substrate retaining spores, germinated spores, and/or growing organism is to be introduced into an environment that is deficient in essential nutrients, all required nutrients can be included in the nutrient phase. Where a cultivation substrate retaining spores/germ inated spores/growing organisms is to be introduced into an environment having at least one essential nutrient, those environmentally-available essential nutrients may be excluded from the nutrient phase or included at a lower concentration. The cultivation substrate may also act to concentrate nutrients from the environment by capturing the environmental nutrients in, for example, the microstructure of the high porosity substrate. This may be advantageous in environments where environmental nutrients are present only in low concentrations.

In some embodiments, and as further described elsewhere herein, the cultivation system can be used to transport retained spores/germinated spores from location to another. Where the cultivation system functions as a transportation system, the nutrient phase may include sufficient nutrient levels to viably support the retained spores/germinated spores/growing organisms during transport. In some embodiments the nutrient phase may include sufficient nutrient levels to viably maintain the retained spores/germinated spores/growing organisms post-transport, following introduction of the retained spores/germinated spores/growing organism into a new environment (e.g., the open water).

In some embodiments the nutrient phase includes one or more carriers. Carriers can include, for example, liquid carriers, gel carriers, and hydrogel carriers. In some embodiments, a carrier of the nutrient phase is an adhesive. Including an adhesive as a carrier of the nutrient phase can function to ensure that the nutrient phase remains on and/or within the cultivation substrate. Where the nutrient phase is applied to a surface of the cultivation substrate and includes an adhesive as a carrier, the nutrient face may also function to promote attachment to the cultivation substrate.

In some embodiments, the nutrient phase is formulated to control release rates of the nutrients.

In some embodiments, the cultivation substrate further comprises a salt associated with the cultivation substrate. In some embodiments, the salt is sodium chloride (NaCl). Salt associated with the cultivation substrate can produce and maintain a saline microenvironment for the retained spores/germinated spores. This can be particularly advantageous when seaweed and marine plants are retained by the cultivation substrate. In some embodiments, a saline microenvironment within the cultivation substrate can be maintained when the cultivation substrate is submerged in fresh water, thereby viably maintaining marine species and avoiding the need to maintain a saline culture environment, which can be difficult and costly.

In some embodiments, the cultivation substrate includes a liquid-containing phase associated with at least a portion of the cultivation substrate. The liquid-containing phase serves to provide and maintain moisture within the microenvironment of the high porosity substrate’s microstructure, which may be beneficial to the viable maintenance of the spores/germinated spores/growing organism retained by the cultivation substrate.

In some embodiments, the cultivation substrate includes a liquid wicking material. The liquid wicking material can be the same material that forms the low porosity substrate and/or the high porosity substrate. The liquid wicking material functions to maintain moisture within the cultivation substrate’s microenvironment.

While spores and endospores may be viably maintained in an arid environment, the germinated spores and growing organisms (e.g., juvenile seaweed) will generally require moisture to grow and/or proliferate. By maintaining a moist microenvironment (e.g., by including a liquid-containing substrate and/or a liquid wicking material), it may be possible to transport the culture system having spores/germinated spores/growing organisms retained therein and/or thereon without having to maintain the cultivation system in an aqueous environment.

In some embodiments, the liquid containing phase is entrained in the low porosity substrate, on the fibrillated submicron surface structure of the low porosity substrate, within the microstructure of the high porosity substrate, on the microstructure (e.g., on its surface) of the high porosity substrate, or any combination thereof. In some embodiments, the liquid containing phase is applied to a surface of the cultivation substrate as a coating. In some embodiments, the liquid containing phase is included within one or more materials forming the cultivation substrate.

In some embodiments, the liquid containing phase includes, for example, a hydrogel, a slurry, a paste, or a combination of a hydrogel, a slurry, and/or a paste. In some embodiments, the liquid containing phase is a carrier for the nutrient phase.

In some embodiments, at least a portion of the cultivation substrate is hydrophilic. Such hydrophilic portions of the cultivation substrate may contribute to retention by the cultivation substrate and/or attachment to the cultivation substrate.

In some embodiments, at least a portion of the cultivation substrate is hydrophobic. Such hydrophobic portions of the cultivation substrate may reduce or prevent or resist retention and/or attachment of spores/germinated spores/growing organisms. This may help reduce or prevent biofouling and attachment of unwanted spores or other cells or organisms to the cultivation substrate.

In some embodiments, one or more portions of the cultivation substrate is hydrophobic, and one or more portions of the cultivation substrate is hydrophilic, such that spores/germinated spores/growing organisms are selectively encouraged to be retained by or attach to the one or more hydrophilic portions of the cultivation substrate.

In some embodiments, the cultivation substrate may include one or more bioactive agents associated with the cultivation substrate. Bioactive agents include any agent having an effect, whether positive or negative, on the cell or organism coming into contact with the agent. Suitable bioactive agents may include, for example, biocides and serums. Biocides may be associated with portions of the cultivation substrate to prevent attachment and growth of unwanted cells or organisms to those portions of the cultivation substrate. Unwanted cells may include non-target cells such as bacteria, yeast, and algae, for example (i.e., biofouling species). Biocides may also deter pests, such as insects. In some embodiments, the biocide prevents attachment and growth of the target spore to portions of the cultivation substrate where attachment and growth is not desired. In some embodiments, serums may be applied to portions of the cultivation substrate. Serums may aid in spore attachment and retention and/or encourage germination of or growth from the spore. Serums may include cell-adhesive ligands, for example, as well as provide a source of growth factors, hormones, and attachment factors.

In some embodiments, the cultivation substrate is patterned. By patterning the cultivation substrate, it is possible to designate areas of the cultivation substrate to which a target spore/germinated spore/growing organism (e.g., juvenile seaweed) with attach. In some embodiments, the cultivation substrate includes a pattern of sections of low porosity substrate and sections of high porosity substrate. In some embodiments, the cultivation substrate is patterned in a “checkerboard” manner, with alternating sections of low porosity substrate and high porosity substrate. The pattern of low porosity substrate and high porosity substrate can be an organized or selective pattern, or it can be a random pattern. The sections of substrate can be all of the same size, or of different sizes. Sections of either the low porosity substrate or the high porosity substrate can be the same size, but different from the other (i.e., all sections of low porosity substrate are the same, but are a different size than the sections of high porosity substrate).

In some embodiments, the fibrillated submicron surface structure of the low porosity substrate and/or the microstructure of the high porosity substrate is patterned. By specifically patterning the fibrillated submicron surface structure, the microstructure, or both, it is possible to specifically retain target spores at described portions of the microstructure while excluding cells from other portions.

In some embodiments, the fibrillated submicron surface structure includes a pattern of differing surface structures. For example, the average inter-fibril distance can be varied across the cultivation substrate. In some embodiments, the low porosity substrate includes a pattern of greater inter-fibril distance portions and lower inter-fibril distance portions. In such embodiments, the differences in inter-fibril distance can promote attachment and holdfast development of different seaweed species. In other embodiments, the fibrillated submicron surface structure in some areas can be eliminated, leaving a smooth surface to which seaweed will not attach. In such embodiments, this allows for controlling where seaweed will attach on the cultivation substrate, and in particular, on the low porosity substrate.

In some em bodim ents, the depth of the fibrillated submicron surface structure can be varied across the cultivation substrate. In some embodiments, the low porosity substrate includes a pattern of greater fibrillated submicron surface structure depth portions and lower fibrillated submicron surface structure depth portions. In such embodiments, the differences in the depth of the fibrillated submicron surface structure can promote attachment and holdfast development of different seaweed species.

In certain embodiments, both the inter-fibril distance and the depth of the fibrillated submicron surface structure can be varied. In such embodiments, the fibrillated submicron surface structure can be finely tuned for a given application.

In some embodiments, the microstructure of the high porosity substrate included in the cultivation substrate includes a pattern of higher density portions and lower density portions. In such a configuration, the lower density portions correspond to a portion of the microstructure configured to retain and viably maintain the target spores, while the higher density portions inhibit or prevent retention of cells. The density pattern may extend in any dimension. For example, a high-density/low-density pattern may extend in the x- or y-dimension of the cultivation substrate, or in the z-dimension. When extending in the z-dimension, the outermost portion will generally be a lower density portion configured to retain and viably maintain the target spores. Underlying portions may be of a higher density, or may be of an even lower density than the outermost portion. Where the underlying portion is of a higher density, ingrowth of a germinated spore will be inhibited or prevented. Where the underlying portion is of a lower density than the outermost portion, ingrowth of the germinated spores will be encouraged and/or facilitated. In some embodiments, the density pattern or gradient in the z-dimension results from concentric wraps of microstructure material having differing densities, or from a laminate configuration in which each lamina has a different density. In some embodiments, the density pattern can extend in two or all three dimensions. In some embodiments, portions of the microstructure have a density gradient.

Density can be measured in various ways, including, for example, measuring dimensions and weight of the material. In addition, wetting experiments can be conducted to derive density values. Density can be modified by, for example, altering inter-fibril distance, number of fibrils per unit volume, number of pores per unit volume, and pore size.

In some embodiments, the density of the high porosity substrate is that of the material itself that forms the high porosity substrate; i.e., does not have any inclusions such as a nutrient phase, liquid containing phase, etc.

In some embodiments, the density of the high porosity substrate is that of the material of the high porosity substrate and an inclusion such as a nutrient phase, a liquid containing phase, or a density-altering filler. In some embodiments, portions of the microstructure are filled with a filler to alter the density, thereby altering the ability of that portion of the microstructure to retain spores and/or prevent ingrowth into the microstructure of the high porosity substrate.

In some embodiments, the high porosity substrate has a pattern of higher porosity portions and lower porosity portions. In some embodiments, the lower porosity portions correspond to portions of the high porosity substrate configured to retain and viably maintain the target spores. In some embodiments, the higher porosity portions correspond to portions of the microstructure configured to retain and viably maintain the target spores.

In some embodiments, the high porosity substrate includes a pattern of greater inter-fibril distance portions and lower inter-fibril distance portions. In some embodiments, the lower inter-fibril distance portions correspond to the portions of the microstructure configured to retain and viably maintain the spores. In such embodiments, the higher inter-fibril distance portions have inter-fibril distances too great to retain the target spores. In other embodiments, the higher inter-fibril distance portions correspond to the portions of the microstructure configured to retain and viably maintain the spores. In such embodiments, the lower inter-fibril distance portions have inter-fibril distances too small to retain the target spores.

In some embodiments, the pattern of the high porosity substrate is generated by controlling at least two of density, porosity, and average inter-fibril distance. In some embodiments, the pattern of the high porosity substrate, whether involving density, porosity, average inter-fibril distance, or a combination thereof, may be an organized or selective pattern, or may be a random pattern.

In some embodiments, the pattern of the high porosity substrate can be set or adjusted by selective application of longitudinal tension. Setting or adjusting the pattern by application of longitudinal tension allow for one to alter the pattern mechanically. In some embodiments, a pattern is set or adjusted in fibrillated, high porosity material by selective application of longitudinal tension.

In some embodiments, a patterned high porosity substrate includes portions that have two or more characteristics favorable to spore retention. For example, a patterned high porosity substrate can have portions of low-density (i.e., about 1.0 g/cm³ or less) and an average inter-fibril distance selected to retain the target spores (e.g., about 30 µm for dulse spores). These same portions may further be hydrophilic and/or include one or more of a nutrient phase, an adhesive, and a bioactive agent. The density, inter-fibril distance, hydrophobicity, nutrient phase, adhesive, and bioactive agent, for example, may each be selected to preferentially retain a target spore.

In some embodiments, the cultivation substrate is configured as a fiber, a membrane, a woven article, a non-woven article, a braided article, a fabric, a knit article, a particulate dispersion, or combinations of these.

In some embodiments, the cultivation system includes at least one of a backer layer, a carrier layer, a laminate of a plurality of layers, a composite material, or combinations of these. The cultivation substrate (i.e., the low porosity substrate and/or the high porosity substrate) can be deposited on the backer layer or carrier layer, or included in a laminate. The backer layer can be, for example, a rope or metal cable. For example, where the cultivation substrate retains and viably maintains seaweed spores, the cultivation substrate can be deposited on a rope or metal cable to produce a seed rope, eliminating the need to wrap a seed string around the rope in the field for open water rope cultivation of seaweed.

In some embodiments, the cultivation substrate has sufficient strength to be moved as a conveyor belt through various growth stages of the retained spores, including harvest of the germinated spores. In some embodiments, the cultivation substrate is deposited on a backer layer, carrier layer, or formed into a laminate to produce a cultivation system having sufficient strength to be moved as a conveyor belt through various growth stages of the retained spores, including harvest of the germ inated spores.

In some embodiments, the cultivation substrate is configured as a particulate dispersion. The fibrillated submicron surface structure of the low porosity substrate and microstructure of the high porosity substrate, when preset, are provided by a plurality of particles in a dispersion formulated for deposition onto a backer layer or a carrier substrate to form the cultivation system. The particles can be, for example, shredded or otherwise fragmented pieces of a fiber, a membrane, a woven article, a non-woven article, a braided article, a fabric, or a knit article having a fibrillated submicron surface structure or microstructure as described herein. In some embodiments, spores are contacted with the particles prior to deposition onto a backer layer or carrier substrate. In other embodiments, spores are contacted with the particles following deposition onto the backer layer or carrier substrate. The particulate dispersion may be deposited onto the backer layer or carrier substrate by, for example, spraying, dip-coating, brushing, or other coating means. In embodiments in which spores contacted with the particles prior to deposition onto a backer layer or carrier substrate, care must be taken to ensure that the deposition method does not negatively affect the retained spores. Spores and endospores may be more resilient and capable of withstanding deposition in such a manner.

In some embodiments, the cultivation substrate comprises an expanded fluoropolymer. In some embodiments, the expanded fluoropolymer is selected from the group of expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), expanded polytetrafluoroethylene (ePTFE), and modified ePTFE. Examples of suitable expanded fluoropolymers include fluorinated ethylene propylene (FEP), porous perfluoroalkoxy alkane (PFA), polyester sulfone (PES), poly (p-xylylene) (ePPX) as taught in U.S. Pat. Publication No. 2016/0032069, ultra-high molecular weight polyethylene (eUHMWPE) as taught in U.S. Pat. No. 9,926,416 to Sbriglia, ethylene tetrafluoroethylene (eETFE) as taught in U.S. Pat. No. 9,932,429 to Sbriglia, polylactic acid (ePLLA) as taught in U.S. Pat. No. 7,932,184 to Sbriglia, et al., vinylidene fluoride-co-tetrafluoroethylene or trifluoroethylene [VDF-co-(TFE or TrFE)] polymers as taught in U.S. Pat. No. 9,441,088 to Sbriglia.

In some embodiments, the expanded fluoropolymer includes the nutrient phase. This may be achieved by co-blending the nutrient phase with the fluoropolymer resin prior to extrusion and expansion of the fluoropolymer.

In some embodiments, the cultivation substrate comprises an expanded thermoplastic polymer. In some embodiments, the expanded thermoplastic polymer forms the microstructure of the cultivation substrate. In some embodiments, the expanded thermoplastic polymer is selected from the group of expanded polyester sulfone (ePES), expanded ultra-high-molecular-weight polyethylene (eUHMWPE), expanded polylactic acid (ePLA), and expanded polyethylene (ePE).

In some embodiments, the cultivation substrate comprises an expanded polymer. In some embodiments, the expanded polymer forms the microstructure of the cultivation substrate. In some embodiments, the expanded polymer is expanded polyurethane (ePU).

In some embodiments, the expanded polymer includes the nutrient phase. This may be achieved by co-blending the nutrient phase with the fluoropolymer resin prior to expansion of the polymer.

In some embodiments, the cultivation substrate comprises a polymer formed by expanded chemical vapor deposition (CVD). In some embodiments, the polymer formed by expanded CVD forms the microstructure of the cultivation substrate. In some embodiments, the polymer formed by expanded CVD is polyparaxylylene (ePPX).

It will be recognized that certain of these materials are better suited for use as one of the low porosity or high porosity substrates. In other embodiments, the low porosity and high porosity substrates are formed from the same type of material, although the material may be processed differently to provide for the various fibrillated submicron surface structures and microstructures described herein.

In some embodiments, the material forms or can be processed to form the fibrillated submicron surface structure. In some embodiments, the fibrillated submicron surface structure is produced by densifying and stretching the expanded fluoropolymer, expanded thermoplastic polymer, expanded polymer, or ePPX. An example of a suitable densified fluoropolymer material for use as the low porosity substrate is taught by U.S. Pat. No. 7,521,010 to Kennedy, the contents of which are hereby incorporated by reference in their entirety. Kennedy teaches a densified fluoropolymer article having a water vapor permeation of about 0.015 g-mm/m²/day or less, and a matrix tensile strength of at least 10,000 psi in two orthogonal directions. The articles are made by compressing expanded porous PTFE at pressures, temperatures, and times which result in near complete elimination of the pores, and subsequent stretching above the crystalline melt temperature. The stretching steps results in a densified ePTFE sheet having greater tensile strength in the direction of stretch than the compressed precursor from which it was made.

As taught by Kennedy, sheets, or films, of ePTFE were produced in accordance with the teachings of U.S. Pat. No. 3,953,566. The ePTFE films are then compressed in accordance with the teachings of U.S. Pat. No. 5,374,473. The densified films are then stretched at temperatures exceeding the crystalline melt temperature of PTFE. Stretch ratios as has as 12:1 at stretch rates of, for example, 5% per second. The stretching process can be done in either direction, both directions either sequentially, or simultaneously, utilizing a pantograph machine or continuously on a tenter from or similar machine. The thickness of the compressed precursor directly impacts the ability to achieve high stretch amounts, as when the compressed precursor is stretched at a temperature above the crystalline melt temperature of the ePTFE, the bulk density increases. The stretching results in a reduction in unit weight and thickness. A significant increase in the matrix tensile strength of the sheet or sheets is also observed. The result of the densifying and stretching procedure is an extremely thin, high PTFE bulk density film and low porosity with extraordinary water vapor permeation coefficients and high tensile strengths in both the x and y directions. In some embodiments, the ePTFE film is sintered prior to the densifying step. Further, the biaxially ePTFE film can include two or more plies of ePTFE. The process can be carried out in a continuous manner.

In some embodiments, a similar process can be applied to other expanded fluoropolymers, expanded thermoplastic polymers, expanded polymers, or ePPX, generating densified membranes having fibrillated submicron surface structures usable as low porosity substrates in the cultivation substrates described.

In certain embodiments, the densified membranes are stretched at temperatures that are lower than the crystalline melt temperature of expanded fluoropolymer films. In some embodiments, densified membranes are stretched at a temperature just below the crystalline melt temperature. The inter-fibril distance and general morphology of the fibrillated submicron surface structure can be controlled through the stretch rate stretch temperature. In some embodiments, the stretch rate and temperature are selected to produce a node and fibril nanostructure, wherein the fibrils are interconnected via nodes. In other embodiments, the stretch rate and temperature are selected to minimize or eliminate the generation of nodes.

In certain embodiments, a fibrillated submicron surface structure occurs only on one side of the low porosity substrate, providing for seaweed attachment only on the one side having the submicron surface structure. In some embodiments, the side lacking the submicron surface structure is bound to, for example, a backing layer.

Depth of the fibrillated submicron surface structure can be controlled via the densifying and stretching steps.

In some embodiments, the expanded fluoropolymer forms the microstructure of the cultivation substrate.

In some embodiments, the cultivation systems described herein can be used in the farming of seaweed. Seaweed spores are contacted for a sufficient time and under predetermined conditions with a cultivation substrate having desired properties for retaining and viably maintaining the spores until at least some of the spores germinate and are retained (i.e., attached) by the cultivation substrate. In some embodiments, the cultivation substrate can be incubated in a medium conducive to the germination of the spores and growth of the germinated spores. In other embodiments, the culture system itself provides a microenvironment conducive to the germination of spores and growth of the germinated spores, at least for a period of time (e.g., during tem porary transport).

In some embodiments, the cultivation substrates described herein can be used as a growth substrate for multicellular organisms from spores. For example, the cultivation substrates can be used to support growth of seaweed from spore to mature seaweed. In some embodiments, the spore that is to mature into the multicellular organism is contacted for a sufficient time and under predetermined conditions with a cultivation substrate, until at least some of the spores germinate and are retained by the cultivation substrate.

In some embodiments, seaweed spores are introduced onto the cultivation substrate, and gametophytes and sporophytes are allowed to mature in a manner similar to traditional culture strings, by depositing the culture substrate either with or without spores retained therein) on a rope, cable, or other support in the field, the traditional step of wrapping a culture string around a rope line can be skipped. This can be accomplished where the culture substrate is provided by a plurality of particles in a dispersion.

In other embodiments, seaweed sporophytes and/or gametophytes are directly introduced onto the culture substrate. Such direct seeding can reduce the laboratory time required to produce a culture string relative to spore seeding.

Culture strings are traditionally maintained and cultured in a laboratory environment using sterilized sea water. The present cultivation systems, through inclusion of sufficient salt within the microstructure of the high porosity substrate, circumvents the need for the expensive and cumbersome systems required for circulation of sterilized sea water by providing a saline microenvironment within the microstructure. In some embodiments, the seeded cultivation substrate is maintained in a standard seaweed cultivation tank, where nutrients are delivered via sterile seawater. By including a nutrient phase sufficient to support seaweed growth, the need to provide external nutrients to the growing seaweed may be obviated.

Generally, culture strings must be carefully transported in sea water while avoiding jostling to prevent gametophyte and sporophyte detachment from the string. The presently described cultivation systems allow for the gametophytes and sporophytes to be safely transported without sea water. This is achievable by the inclusion of salt and a liquid containing phase within the microstructure, which provides a saline microenvironment having sufficient moisture to support the juvenile seaweed during transport.

In some embodiments, by controlling the attachment strength of the seaweed to the fibrillated submicron surface structure, it is possible to reuse the cultivation substrate or parts thereof, such as the low porosity substrate. By controlling the fibrillated submicron surface structure, it may be possible to provide for sufficiently strong attachment to allow for farming, but not so strong that the holdfasts cannot be mechanically removed by, for example, power washing. After removing the attached seaweed, the low porosity substrate can be reused.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A cultivation system comprising a cultivation substrate including a low porosity substrate having a porosity of about 10% or less, and a fibrillated submicron surface structure configured to retain seaweed by a holdfast.

2. The cultivation system of claim 1, wherein the fibrillated submicron surface structure is characterized by an average inter-fibril distance up to and including 1000 nm.

3. The cultivation system of claim 1, wherein the fibrillated submicron surface structure has an average depth of about 1000 nm or less.

4. (canceled)

5. The cultivation system of claim 1, wherein the low porosity substrate is about 25.4 µm (1 mil) to about 762 µm (30 mil) thick.

6. (canceled)

7. The cultivation system of claim 1, wherein the cultivation substrate is configured as a tape, a substrate, a woven article, a non-woven article, a braided article, a knit article, a fabric, a particulate dispersion, or combinations of two or more of the foregoing.

8. The cultivation system of claim 1, wherein the cultivation substrate includes at least one of a backer layer, a carrier layer, a laminate of a plurality of layers, a composite material, or combinations thereof.

9. The cultivation system of claim 1, wherein the low porosity substrate comprises an expanded fluoropolymer.

10. The cultivation system of claim 9, wherein the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), and expanded polytetrafluoroethylene (ePTFE).

11. The cultivation system of claim 1, wherein the low porosity substrate is an expanded polytetrafluoroethylene (ePTFE) substrate.

12. The cultivation system of claim 1, wherein the low porosity substrate comprises an expanded thermoplastic polymer.

13-16. (canceled)

17. The cultivation system of claim 11, wherein the PTFE substrate has a water vapor permeability coefficient of about 0.015 g-mm/m²/day or less, and is formed by a method comprising: (a) preparing a biaxially expanded PTFE film; (b) densifying the expanded PTFE film; and (c) stretching the densified expanded PTFE film.

18. The cultivation system of claim 17, wherein in step (c), the densified expanded PTFE film is stretched at a temperature exceeding the crystalline melt temperature of PTFE.

19. The cultivation system of claim 17, wherein the expanded PTFE film is sintered prior to step (b).

20. (canceled)

21. The cultivation system of claim 17 wherein steps (a)-(c) are carried out in a continuous manner.

22-35. (canceled)

36. The cultivation system of claim 1, further comprising a nutrient phase associated with at least a portion of the cultivation substrate.

37. The cultivation system of claim 36, wherein the nutrient phase promotes growth of the seaweed and/or attachment of the seaweed to the cultivation substrate.

38. The cultivation system of claim 36, wherein at least a portion of the nutrient phase is entrained within the cultivation substrate, entrained on the cultivation substrate, or entrained within and on the cultivation substrate.

39. The cultivation system of claim 36, wherein the nutrient phase is present as a coating on a surface of the cultivation substrate.

40-41. (canceled)

42. A method for cultivating seaweed, comprising contacting a population of seaweed gametophytes and/or sporophytes with the cultivation substrate of the cultivation system of claim 1 until at least a portion of the population of seaweed gametophytes and/or sporophytes form a holdfast to the nanostructure of the cultivation substrate.

43. The method of claim 25, further comprising positioning the cultivation system in an open-water environment after the portion of the population of seaweed gametophytes and/or sporophytes form a holdfast to the nanostructure of the cultivation substrate.
